(19) United States
(12) Patent Application Publication
(10) Pub. No.: US 2015/0133560 A1
(43) Pub. Date: May 14, 2015

(54) N-Methyl-N-Acylglucamine-Containing Composition

(71) Applicants: Peter Klug, Grossostheim (DE); Carina Mildner, Frankfurt am Main (DE)

(72) Inventors: Peter Klug, Grossostheim (DE); Carina Mildner, Frankfurt am Main (DE)

(73) Assignee: Clariant Finance (BVI) Limited, Tortola (VG)

(21) Appl. No.: 14/401,315

(22) PCT Filed: May 29, 2013
(86) PCT No.: PCT/EP2013/061105

§ 371 Date: Nov. 14, 2014

(30) Foreign Application Priority Data
May 30, 2012 (DE) 10 2012 010 660.2

(51) Int. Cl. A61K 8/42; A61K 8/46; A61K 8/34

(52) U.S. Cl. CPC A61K 8/42; A61K 8/345; A61K 8/463; A61K 2800/10
USPC 514/625

(57) ABSTRACT

The invention relates to a surfactant concentrate which contains at least one anionic surfactant, an N-methyl-N-acylglucamine, a solvent and optionally one or more additives. The invention also relates to a method for producing the surfactant concentrate. The invention further relates to a method for producing cosmetic, dermatological or pharmaceutical compositions.

The invention relates to a surfactant concentrate containing at least one anionic surfactant, an N-methyl-N-acylglucamine, a solvent and if desired one or more additives, and also a process for producing the surfactant concentrate. The invention further relates to a process for producing cosmetic, dermatological or pharmaceutical compositions.

EP 0 550 637 B1 describes a process for producing polyhydroxy fatty acid amide materials which can, inter alia, be used as surfactants. The present process is particularly useful when the N-alkylpolyhydroxyamine has the formula N(R¹)CH₂(CH₂OH)₄CH₂OH. The fatty acid ester type which is preferably used in the process is a C₁₂-C₂₀ fatty acid methyl ester. A preferred process for producing laundry detergent surfactants is one in which the N-alkylpolyhydroxyamine is an N-methylglucamine, the fatty acid ester is a C₁₂-C₂₀-methyl ester or a mixture thereof, the solvent is methanol and the catalyst is sodium methoxide.

However, the above polyhydroxy fatty acid amides have the disadvantage that they are high-melting solids as water-free surfactants. Furthermore, especially N-methyl-N-acylglucamines which have a C₁₂- and C₁₄-acyl radical or a C₁₆- and C₁₈-acyl radical easily form gels on dilution with water and these gels make the handling of the products much more difficult.

EP 0 780 464 A2 relates to a process for producing light-colored, low-viscosity surfactant concentrates by mixing sugar surfactants and betaines in the gel phase. For this purpose, EP 0 780 464 A2 describes a process for producing light-colored, low-viscosity surfactant concentrates, in which

(a1) alkyl and/or alkenyl oligoglycosides and/or

(a2) N-alkylpolyhydroxyalkylamides of fatty acids and

(b) betaine surfactants are mixed in a weight ratio of a:b of from 90:10 to 10:90, with the proviso that the starting materials are present in the gel phase.

It has been found that in order to produce the desired concentrates it is not necessary to start out from low-viscosity, i.e. diluted, aqueous starting materials and concentrate these subsequently. Rather, it has been found that mixing of concentrated starting materials which are present in the gel phase and thus themselves do not have a low viscosity gives products which have a low viscosity and are light-colored and storage-stable. However, EP 0 780 464 A2 does not describe any inventive examples using N-acyl-N-alkylglucamine or any alkyl ether sulfate, which is still the most widely used and most economical surfactant in the cosmetics market.

It is therefore an object of the invention to provide an improved, economical surfactant concentrate which overcomes the disadvantages of the prior art, in particular to provide a concentrate of a surfactant which offers improved handling for further processing.

The invention accordingly provides a surfactant concentrate containing:
(A) 30-60% by weight of at least one anionic surfactant as component A,
(B) 10-40% by weight of at least one N-methyl-N-acylglucamine as component B, where the N-methyl-N-acylglucamine has a C₈- C₂₂-acyl radical,
(C) 10-60% by weight of at least one solvent as component C and
(D) 0-5% by weight of one or more additives as component D, where the sum of the components A to D is 100% by weight.

It has been found that the surfactant concentrates of the invention have a viscosity comparable to commercial ether sulfate pastes and the surfactant concentrate brings about reduced gel formation compared to ether sulfate paste when diluted with water. As a result, diluted solutions produced from the surfactant concentrates of the invention are easier to mix with further constituents and homogenize in the formulation process. The compositions obtained, in particular cosmetic, dermatological or pharmaceutical compositions, have no inhomogeneities. In addition, the stirring times in the production of the compositions are reduced.

The surfactant concentrate of the invention preferably has a viscosity at 30° C. of 10 000-50 000 mPas, in particular a viscosity of 15 000-30 000 mPas.

Further names for N-methyl-N-acylglucamine are N-methyl-N-1-deoxysorbitol fatty acid amide, N-acyl-N-methylglucamine, glucamide and N-methyl-N-alkylglucamide. Here, N-methyl-N-acylglucamine corresponds to the formula (X), where R is an organic radical:

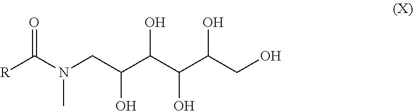

The surfactant concentrate preferably consists of
(A) 35-50% by weight of at least one anionic surfactant as component A,
(B) 20-35% by weight of at least one N-methyl-N-acylglucamine as component B, where the N-methyl-N-acylglucamine has a C₈-C₂₂-acyl radical,
(C) 20-45% by weight of at least one solvent as component C and
(D) 0-2% by weight of one or more additives as component D, where the sum of the components A to D is 100% by weight.

The concentrated surfactant concentrate of the invention advantageously allows dilution of the concentrate even though the individual components, in particular N-methyl-N-acylglucamines, form gels which are sparingly soluble in water. This property can advantageously be achieved by means of the synergistic effect among the components.

In a further preferred embodiment, the surfactant concentrate contains
(A) 45-50% by weight of at least one anionic surfactant as component A,
(B) 20-25% by weight of at least one N-methyl-N-acylglucamine as component B, where the N-methyl-N-acylglucamine has a C₈-C₂₂-acyl radical,
(C) 25-35% by weight of at least one solvent as component C and
(D) 0-2% by weight of one or more additives as component D, where the sum of the components A to D is 100% by weight.

The surfactant concentrate preferably consists of the components A, B and C:
(A) 30-60% by weight of at least one anionic surfactant as component A,
(B) 10-40% by weight of at least one N-methyl-N-acylglucamine as component B, where the N-methyl-N-acylglucamine has a C₈-C₂₂-acyl radical,
(C) 10-60% by weight of at least one solvent as component C.

Due to the high surfactant concentration, the compositions of the invention are self-preserving and do not require any additional preservatives.

In a preferred embodiment, the solvent is a protic solvent.

In a preferred embodiment, the surfactant concentrate consists of the components A, B, C, where the component C consists of water and propylene glycol or water, propylene glycol and glycerol.

In a preferred embodiment, the component A is selected from among one or more compound(s) of the general formula (I),

R¹SO₃⁻M⁺ (I)

where R¹ is alkyl, cycloalkyl, aralkyl, aryl, alkoxy, alkoxyalkyl or heterocylcyl and M⁺ is an alkali metal ion, an alkaline earth metal ion or a substituted or unsubstituted ammoniumion, or

of the general formula (II),

R¹SO₄⁻M⁺ (II)

where R¹ is alkyl, cycloalkyl, aralkyl, aryl, alkoxy, alkoxyalkyl or heterocyclyl and M⁺ is an alkali metal ion, an alkaline earth metal ion or a substituted or unsubstituted ammonium ion.

“Alkyl” is a saturated aliphatic hydrocarbon group which can be linear or branched and can have from 1 to 20 carbon atoms in the chain. Preferred alkyl groups can be linear or branched and have from 1 to 10 carbon atoms in the chain. Branched means that a lower alkyl group such as methyl, ethyl or propyl is present as substituent on a linear alkyl chain. Alkyl is, for example, methyl, ethyl, 1-propyl, 2-propyl, 1-butyl, 2-butyl, 2-methyl-1- propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl; 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl.

“Cycloalkyl” is an aliphatic ring which has from 3 to 10 carbon atoms in the ring. Preferred cycloalkyl groups have from 4 to 7 carbon atoms in the ring.

“Aryl” is phenyl or naphthyl.

“Aralkyl” is an alkyl group which is substituted by an aryl radical.

“Substituted aralkyl” and “substituted aryl” mean that the aryl group or the alkyl group of the aralkyl group is substituted by one or more substituents selected from among alkyl, alkoxy, nitro, carboalkoxy, cyano, halo, alkylmercaptyl, trihaloalkyl or carboxyalkyl.

“Alkoxy” is an alkyl-0 group in which “alkyl” is as defined above. Lower alkoxy groups are preferred. Examples are methoxy, ethoxy, n-propoxy, i-propoxy and n-butoxy.

“Lower alkyl” is an alkyl group having from 1 to 7 carbon atoms.

“Alkoxyalkyl” is an alkyl group as described above which is substituted by an alkoxy group as described above. The term alkoxyalkyl can thus encompass a polyether.

“Heterocyclyl” is a 4- to 10-membered ring structure in which one or more ring atoms are not carbon, for example are N, O or S. Heterocyclyl can be aromatic or nonaromatic, i.e. it can be saturated, partially unsaturated or fully unsaturated.

In a preferred embodiment, the anionic surfactant is selected from the group consisting of sodium lauryl ether sulfate, laurylsulfate, cocosulfate and mixtures thereof.

Particular preference is given to sodium cocosulfate or sodium lauryl ether sulfate.

In a preferred embodiment, the acyl radical in the component B is selected from the group consisting of linear or branched, saturated or unsaturated C₈-C₂₂-acyl radicals and mixtures thereof.

In a preferred embodiment, the component B consists of various N-methyl-N-acylglucamines, where the respective acyl radicals are different and the acyl radicals are derived from carboxylic acids selected from the group consisting of oleic acid, linoleic acid, caprylic acid, capric acid, lauric acid, myristic acid, palmitic acid and stearic acid.

For the purposes of the invention, a solvent is preferably a protic solvent such as water, a C₁-C₈-alcohol, in particular C₁-C₆-alcohol, ethylene glycol, diethylene glycol, triethylene glycol, propylene glycol, glycerol or a mixture thereof, with particular preference being given to water, water and propylene glycol and also water and propylene glycol and glycerol. Among C₁-C₆-alcohols, preference is given to methanol, ethanol, isopropanol, n-butanol and sec-butanol.

In a preferred embodiment, the sum of the components A and B is from 50 to 80% by weight, preferably from 60 to 75% by weight and in particular from 65 to 70% by weight.

In a preferred embodiment, the component B comprises from 2 to 6 different N-methyl-N-acylglucamines.

In a preferred embodiment, the component B comprises from 2 to 6 different N-methyl-N-acylglucamines, with the 2 to 6 different N-methyl-N-acylglucamines having acyl radicals having different even numbers of carbon atoms. This means, for example, that the acyl radicals of two N-methyl-N-acylglucamines are derived from octanoic acid and tetradecanoic acid.

In a preferred embodiment, the component B consists of two N-methyl-N-acylglucamines and the acyl radicals differ by not more than two carbon atoms. When, for example, the acyl radical of an N-methyl-N-acylglucamine is a C₁₆-acyl radical (N-methyl-N-C₁₆-acylglucamine), the other acyl radical is a C₁₈-acyl radical (N-methyl-N-C₁₈-acylglucamine).

In a preferred embodiment, the component B consists of a mixture of N-methyl-N-C₁₂-acylglucamine and N-methyl-N-C₁₄-acylglucamine.

In a preferred embodiment, the additives are selected from the group consisting of complexing agents, cationic polymers, activity reinforcers, acids, alkalis, preservatives, fragrances, dyes, further surfactants, oil bodies, oiling agents, moisture-donating agents, stabilizers and mixtures thereof, preferably in amounts of from 0 to 5.0% by weight, particularly preferably from 0 to 2.0% by weight and in particular from 0.1 to 1.0% by weight.

Suitable preservatives are the preservatives listed in the relevant annex of the European Cosmetics Legislation, for example phenoxyethanol, benzyl alcohol, parabens, benzoic acid and sorbic acid; 1,3-bis(hydroxymethyl)-5,5-dimethylimidazolidine-2,4-dione (Nipaguard® DMDMH), for example, is particularly useful.

In a preferred embodiment, the concentrate is a concentrate for cosmetic, dermatological or pharmaceutical compositions.

The invention further provides a process for producing the concentrate of the invention, which comprises the steps:
a) mixing of the components B and C, during which the mixture is preferably heated to 70-90° C.,
b) addition of the mixture from a) to component A, with the temperature of the component A preferably being 70-90° C.,
c) homogenization of the mixture of A, B and C from c) and
d) if desired addition of the component E.

The process preferably comprises the abovementioned amounts of the surfactant concentrate of the invention.

The invention further provides a process for producing cosmetic, dermatological or pharmaceutical compositions, wherein the surfactant concentrate of the invention is diluted with a solvent. Water is preferably used as solvent.

The invention further provides for the use of the surfactant concentrate of the invention as cleaner. Here, the surfactant concentrate is diluted with water.

The invention is illustrated by the following examples:

Production examples H1 and H2 and examples 1 and 2.

The N-acyl-N-methylglucamines described below were prepared from the corresponding fatty acid methyl esters and N-methylglucamine in the presence of 1,2-propylene glycol as solvent as described in EP 0 550 637 and obtained as solid consisting of active substance and 1,2-propylene glycol.

TABLE 1

| | | Active | 1,2- | |
|---|---|---|---|---|
| Production | Methyl | substance | propylene | Melting |
| example | ester | (%) | glycol (%) | point |
| H1 | C12/14 | 90 | 10 | 85 |
| H2 | C16/18 | 80 | 20 | 65 |

Production of the concentrated surfactant compositions of the invention: The N-acyl-N-methylglucamines from production examples H1 and H2 were stirred with water at 80° C. The resulting solution was stirred into 70% strength (sodium) lauryl ether sulfate paste (Genapol LRO paste), which had likewise been heated to 80° C., and homogenized.

Composition of the surfactant compositions of the invention:

TABLE 2

| | | | | | Active |
|---|---|---|---|---|---|
| | | Lauryl ether | | Active | content of |
| | Production | sulfate paste | Water | content of | lauryl ether |
| Example | example | 70% (%) | (%) | glucamide | sulfate |
| Example 1 | Production | 67.1 | 7.3 | 23 | 47 |
| | example | | | | |
| | H1, 25.6% | | | | |
| Example 2 | Production | 67.1 | 4.1 | 23 | 47 |
| | example | | | | |
| | H2, 28.8% | | | | |

012/14 and 016/18 glucamide form gels which are normally sparingly soluble in water on dilution, but C12/14 has advantages in respect of the thickening performance. Mixing with sodium lauryl ether sulfate (SLES) makes it possible to obtain readily dilutable concentrates having advantageous handling.

The inventive compositions of examples 1 and 2 are pumpable concentrates which have a viscosity comparable to lauryl ether sulfate paste (lauryl ether sulfate paste (70%) at 30° C.: 13 000 mPa.s; example 1 at 30° C.: 22 000 mPa.s) and can readily be dissolved in water with stirring. The gel phase of lauryl ether sulfate is thus firstly reduced on dilution, and secondly gel formation of the chain-pure N-acyl-N-methylglucamines in water is suppressed.

The viscosities were measured using a Brookfield viscometer model DV II and the spindles from the spindle set RV at 20 revolutions per minute and 20° C. or 30° C. The spindles 1 to 7 from the spindle set RV are used. Under these measurement conditions, spindle 1 is selected for viscosities of not more than 500 mPa.s, spindle 2 for viscosities of not more than 1000 mPa.s, spindle 3 for viscosities of not more than 5000 mPa.s, spindle 4 for viscosities of not more than 10 000 mPa.s, spindle 5 for viscosities of not more than 20 000 mPa.s, spindle 6 for viscosities of not more than 50 000 mPa.s and spindle 7 for viscosities of not more than 200 000 mPa.s.

1. A surfactant concentrate containing:
(A) 30-60% by weight of at least one anionic surfactant as component A,
(B) 10-40% by weight of at least one N-methyl-N-acylglucamine as component B, where the N-methyl-N-acylglucamine has a C₈-C₂₂-acyl radical,
(C) 10-60% by weight of at least one solvent as component C and
(D) 0-5% by weight of one or more additives as component D, where the sum of the components A to D is 100% by weight.

2. The surfactant concentrate as claimed in claim 1, wherein the solvent is a protic solvent.

3. The surfactant concentrate as claimed in claim 1, wherein the surfactant concentrate consists of the components A, B, C, where component C consists of water and propylene glycol or water, propylene glycol and glycerol.

4. The surfactant concentrate as claimed in claim 1, wherein component A is selected from the group consisting of compound(s) of the general formula (I),
R¹SO₃⁻M⁺ (I)
where R¹ is alkyl, cycloalkyl, aralkyl, aryl, alkoxy, alkoxyalkyl or heterocyclyl and M⁺ is an alkali metal ion, an alkaline earth metal ion or a substituted or unsubstituted ammonium ion, and
of the general formula (II),
R¹SO₄³¹ M⁺ (II)
where R¹ is alkyl, cycloalkyl, aralkyl, aryl, alkoxy, alkoxyalkyl or heterocyclyl and M⁺ is an alkali metal ion, an alkaline earth metal ion or a substituted or unsubstituted ammonium ion.

5. The surfactant concentrate as claimed in claim 1, wherein the anionic surfactant is selected from the group consisting of sodium lauryl ether sulfate, laurylsulfate, cocosulfate and mixtures thereof.

6. The surfactant concentrate as claimed in claim 1 any wherein the acyl radical in component B is selected from the group consisting of linear or branched, saturated or unsaturated C₈-C₂₂-acyl radicals and mixtures thereof.

7. The surfactant concentrate as claimed in claim 1, wherein component B consists of various N-methyl-N-acylglucamines, where the respective acyl radicals are different and the acyl radicals are derived from carboxylic acids selected from the group consisting of oleic acid, linoleic acid, caprylic acid, capric acid, lauric acid, myristic acid, palmitic acid and stearic acid.

8. The surfactant concentrate as claimed in claim 1, wherein the sum of the components A and B is from 50 to 80% by weight.

9. The surfactant concentrate as claimed in claim 1, wherein component B comprises from 2 to 6 different N-methyl-N-acylglucamines.

10. The surfactant concentrate as claimed in claim 1, wherein component B comprises from 2 to 6 different N-methyl-N-acylglucamines, with the 2 to 6 different N-methyl-N-acylglucamines having acyl radicals having different even numbers of carbon atoms.

11. The surfactant concentrate as claimed in claim 1, wherein component B consists of two N-methyl-N-acylglucamines and the acyl radicals differ by not more than two carbon atoms.

12. The surfactant concentrate as claimed in claim 1, wherein the one or more additives are selected from the group consisting of complexing agents, cationic polymers, activity reinforcers, acids, alkalis, preservatives, fragrances, dyes, further surfactants, oil bodies, oiling agents, moisture-donating agents, stabilizers and mixtures thereof.

13. A concentrate for cosmetic, dermatological or pharmaceutical compositions comprising the surfactant concentrate as claimed in claim 1.

14. A process for producing the surfactant concentrate as claimed in claim 1, comprising the steps of:
a) mixing of the components B and C,
b) addition of the mixture from step a) to component A,
c) homogenization of the mixture of A, B and C) and optionally
d) adding component D.

15. A process for producing a cosmetic, dermatological or pharmaceutical composition, comprising the step of diluting the surfactant concentrate as claimed in claim 1 with a solvent.
